(19) United States
(12) Patent Application Publication
(10) Pub. No.: US 2020/0263249 A1
(43) Pub. Date: Aug. 20, 2020

(54) Method and Kit for Predicting Susceptibility to Allergic Reaction

(71) Applicant: Apollonian Biosystems Limited, Palo Alto, CA (US)

(72) Inventors: See-Ying TAM, Palo Alto, CA (US); Yee San Issan TAM, Hong Kong (HK); Hang Yung Alaster LAU, Hong Kong (HK)

(21) Appl. No.: 16/278,200

(22) Filed: Feb. 18, 2019

(51) Int. Cl. C12Q 1/6876; C12Q 1/686; C12Q 1/6806

(52) U.S. Cl. CPC C12Q 1/6876; C12Q 1/686; C12Q 1/6806; G01N 2800/24; C12Q 2600/118; C12Q 2600/112; C12Q 2600/158

(57) ABSTRACT

The present invention relates to a kit for determining susceptibility to an allergic reaction for a subject. The kit includes a probe for identifying a target biomarker, and an instruction that indicates the subject's susceptibility to the allergic reaction. The present invention also relates to a method of predicting susceptibility to an allergic reaction for a subject and treating and/or preventing the allergic reaction.

### FIELD OF INVENTION

This invention relates to a kit for determining the likelihood of an allergic reaction in a subject. The invention also relates to a method of predicting susceptibility to an allergic reaction and treating or preventing the allergic reaction.

### BACKGROUND OF INVENTION

Precision Medicine represents an important emerging trend in the pharmaceutical and health care industries that offers an innovative approach to drug discovery, development and treatment. This approach embraces the outlook that different subsets of therapeutic products need to be developed specifically in order to treat different subsets of patients of the same disease populations because of different genetic backgrounds and different life histories. Precision Medicine thus calls for the development of specific screening and diagnostic approaches based on identification of biomarkers that can allow patient stratification in both clinical trial studies and actual therapeutic treatments.

A biomarker is defined as a characteristic that is objectively measured and evaluated as an indicator of normal biological processes, pathogenic processes or pharmacologic responses to a therapeutic intervention. Common biomarkers include genetic mutations, mRNA expression levels, protein concentrations and other measureable factors. In the context of clinical research and treatments, biomarkers are used for (a) patient pre-selection or stratification (diagnostic, prognostic, predictive) and (b) monitoring response during the trial or treatment.

Allergy is an overreaction of the human immune system to a foreign substance (allergen), which can be represented by a protein or chemical. The symptoms of allergy can range from mild reactions to severe reactions such as anaphylaxis. It is a major health issue in the developed world as it is estimated that 1 in 5 individuals develop some types of allergy in their lifetimes, and allergy prevalence has been increasing since the 1980s across all age, sex and racial groups.

Mast cells are the major effector cells for IgE-dependent and IgE-independent allergic reactions. As part of the initial IgE-mediated allergic response, B cells in the human immune system makes specific IgE antibodies to allergens. These allergen-specific antibodies bind to specific high affinity IgE receptors (FcεRI) expressed on the surface of mast cells, which reside in the skin, lungs, nose and bowels. Upon allergen re-exposure, the antigen-bound IgE causes rapid release of histamine and other mediators from mast cells, resulting in many types of symptoms and reactions associated with hypersensitivity and allergic diseases such as anaphylaxis, food allergy, rhinitis, itch, urticaria, atopic dermatitis, and asthma. On the other hand, mast cells can also be activated via an IgE-independent mechanism. The G-protein-coupled receptor, MRGPRX2, is expressed on human mast cells and mediates the mast cell-activating effects of many drugs and cationic proteins in anaphylactic events.

Allergic reactions are manifested in different manners and in different symptomatic severity, depending on the tissues that are affected, which include skin rashes, sinus congestion, bronchial constriction, abdominal pain, diarrhea, and systemic shock. In the most severe form of systemic allergic reaction, which is called anaphylaxis, mast cell-derived mediators can severely restrict airways, leading to asphyxiation, cardiovascular collapse and eventually to death.

In view of the demand for prediction, treatment and prevention of allergy, improvements in methods and kits therefor are desired.

### SUMMARY OF INVENTION

This present invention relate to kits and methods that predict susceptibility to an allergic reaction for a subject by determining the expression level(s) of one or more target biomarkers.

One example embodiment is a kit for determining susceptibility to an allergic reaction for a subject. The kit includes a probe for identifying a target biomarker from a biological sample of the subject. The kit optionally includes an instruction that indicates the subject's susceptibility to the allergic reaction by comparing an expression level of the target biomarker of the subject with a threshold. The target biomarker is selected from a group consisting of neural EGFL like 2 protein (NELL2), A-kinase anchor protein 12 (AKAP12), integral membrane protein 2C (ITM2C), and interleukin 13 receptor subunit alpha 1 (IL13RA1) and any combination thereof. The threshold is defined as the mean of expression levels of the target biomarker in the biological samples of subjects who have an average risk of the allergic reaction.

Another example embodiment is a method of predicting susceptibility to an allergic reaction for a subject and treating/preventing the allergic reaction. The method includes obtaining a biological sample from the subject; comparing an expression level of the target biomarker of the subject with the threshold; identifying the subject as having a high susceptibility to the allergic reaction if the expression level of NELL2 and/or AKAP12 and/or IL13RA1 and/or ITM2C of the subject is higher than the threshold. The method also includes administering to the subject having the high susceptibility an anti-allergic drug for treatment or prevention of the allergic reaction, or prescribing a diet or treatment regimen wherein the subject avoids exposure to allergens associated with allergic reaction.

Other example embodiments are discussed herein.

### BRIEF DESCRIPTION OF FIGURES

FIG. 1 is histamine release (%) from individual donor mast cell cultures in response to anti-IgE and A23187 stimulations. Anti-IgE: 26.8±15.1%; A23187: 75.3±8.7% (mean±SEM, N=146).

FIG. 2 is distribution of numbers of individual donor cultures with different levels of histamine release (%) in response to anti-IgE stimulation.

FIG. 3 is histamine release (%) in response to anti-IgE and A23187 stimulations for samples selected from the three responder groups [High, Average (Ave), Low] that were comprised in the 50 donor samples selected for microarray analysis.

FIG. 4 Expression levels of four representative genes assessed by TaqMan qPCR studies in comparison of samples that were selected from High, Low, and Average (Ave) responder groups, respectively (N=51). Statistical analysis (two-tailed t-test) revealed significant differences in the comparison of mRNA expression levels of NELL2, IL23RA1, AKAP12 and ITM2X between High responder group vs Average responder group:
NELL2: p=0.0076
IL13RA1: p=0.0289
AKAP12: p=0.0076
ITM2C: p=0.0167.

### DETAILED DESCRIPTION

Example embodiments relate to kits and methods that predict susceptibility to an allergic reaction for a subject by determining the expression level of one or more biomarkers, such as those expressed in mast cells.

Allergy is an abnormal overreaction of the human immune system to foreign substances and is a major health issue in the developed world. It is still unknown why only some people become allergic. Genetic and epigenetic factors are major components that contribute importantly to susceptibility to allergic reactions.

Among the known immune cells, mast cells are the major effector cells of allergy as their activation induced by binding with both allergens and allergen-specific IgE antibodies directly results in many typical allergic symptoms and reactions including the life-threatening anaphylaxis reaction.

As part of the initial allergic response, B cells in the human immune system make specific IgE antibodies to allergens. These allergen-specific antibodies bind to specific high affinity IgE receptors (FcεRI) expressed on the surface of mast cells, which are the major effector cells for allergic reactions that reside in the skin, lungs, nose and bowels. Upon allergen re-exposure, the antigen-bound IgE causes rapid release of histamine and other mediators from mast cells, resulting in many types of symptoms and reactions associated with hypersensitivity and allergic diseases such as anaphylaxis, food allergy, rhinitis, itch, urticaria, atopic dermatitis, and asthma.

Avoiding exposure to an allergen is one of the ways in which to prevent the occurrence of allergy. Example embodiments solve the problem by providing methods and kits that determine a subject's susceptibility to an allergic reaction, so that the subject is able to take precautions before the exposure to the allergen.

In one example embodiment, a kit is provided to determine a subject's susceptibility to an allergic reaction, or likelihood of reaching a certain degree of severity of an allergic reaction. The expression levels of one or more biomarkers in a biological sample of the subject are measured. The susceptibility to or the degree of the severity of the allergic reaction for the subject can be determined based on the measured expression levels of the biomarker(s).

The biomarker(s) are selected from neural EGFL like 2 protein (NELL2), A-kinase anchor protein 12 (AKAP12), integral membrane protein 2C (ITM2C), and interleukin 13 receptor subunit alpha 1 (IL13RA1) or any combination thereof. In one example embodiment, the expression level of the biomarker(s) is measured by determining the expression level of the biomarker gene or the protein. In one example embodiment, the expression levels of any combination of the target biomarkers are measured by quantitative real-time PCR to determine the susceptibility to the allergic reaction.

In one example embodiment, measurement of the expression level of the biomarker(s) does not require the subject to be stimulated by an allergen, such as an allergen which causes Immunoglobulin E (IgE) mediated allergic reaction. Therefore, the kit can be applied to predict the subject's susceptibility to the allergic reaction, even though the subject is not exposed to any allergens during the testing.

The kit includes a probe for identifying the target biomarker(s) from the biological sample of the subject. In one example embodiment, the biological sample is generated from the blood of the subject. In one example embodiment, the biological sample is blood-derived mast cells of the subject. In one example embodiment, the biological sample is mast cells generated by an in-vitro method from the peripheral blood of the subject.

The probe can identify the target biomarker(s) such that the expression level(s) of the target biomarker(s) can be measured. In one example embodiment, the probe is a nucleotide sequence that is complementary to at least one fragment of the gene of the target biomarker so that the probe can identify the biomarker in the biological sample, such as mast cells. In one example embodiment, the probe is a nucleotide sequence that is complementary or identical to a part of the gene of the biomarker.

In another example embodiment, the probe is an antibody specific to the target biomarker. The probe binds to the protein of the biomarker so that the probe can identify the biomarker in the biological sample such as the mast cells. In one example embodiment, the antibody is a monoclonal antibody or a polyclonal antibody. In one example embodiment, the antibody is an antibody from a non-human species.

The kit optionally includes an instruction which shows how to perform a comparison between the expression level of each target biomarker and a threshold, and indicates the subject's susceptibility to the allergic reaction based on the comparison.

In one example embodiment, the expression level of the target biomarker(s) is determined by measuring the expression level of the protein of the biomarker(s). In one example embodiment, the expression level of the target biomarker(s) is determined by measuring the expression level of the gene of the biomarker(s) which encodes the protein.

In one example embodiment, the threshold is a range of expression levels of the target biomarker(s) for subjects who do not have risks of allergy or do not need to avoid contacting allergens to prevent allergy. In one example embodiment, the threshold is generated by obtaining the mean of expression levels of the target biomarker(s) of subjects who have an average risk of allergic reaction.

In one example embodiment, the threshold is defined by the followings: determining levels of histamine release in response to an allergen in a group of sample subjects; calculating a mean value of the levels of the histamine release among the group and optionally a standard deviation value; calculating a range for an average histamine release; and defining the mean of expression levels of the target biomarker of the sample subjects with average histamine release as the threshold. The range for an average histamine release is defined as [mean value−standard deviation value] (lower limit) to [mean value+standard deviation value] (upper limit). Sample subjects whose histamine release levels are lower than the lower limit of the average range are categorized as Low responders. Sample subjects whose histamine release levels are higher than the upper limit of the average range are categorized as High responders. In some embodiments, the range of histamine release levels for mast cells derived from Average responders is 11.7%-41.9%. In some embodiments, the subjects have high risk of susceptibility to the allergic reaction when the histamine releases from mast cells derived from these High responder subjects are higher than 41.9%. In some embodiments, the subjects have low risk of susceptibility to the allergic reaction when the histamine releases from mast cells derived from these Low responder subjects are lower than 11.7%.

In one example embodiment, a user will know whether the tested subject should prevent occurrence of allergy after using the kit. If the expression level of NELL2 and/or AKAP12 and/or IL13RA1 and/or ITM2C in the cells obtained from the tested subject is higher than the threshold, the tested subject has a high susceptibility to the allergic reaction. The instruction will inform the user that the tested subject has a high susceptibility to the allergic reaction and should treat and/or prevent occurrence of the allergic reaction.

In one example embodiment, if the expression level of NELL2 and/or AKAP12 and/or IL13RA1 and/or ITM2C in the cells obtained from the tested subject is lower than the threshold, the tested subject has a low susceptibility to the allergic reaction. The user is then informed that the tested subject has a low susceptibility to the allergic reaction and does not need to treat and/or prevent occurrence of the allergic reaction.

In one example embodiment, if the expression levels of NELL2 and AKAP12 and IL13RA1 and ITM2C of the tested subject are higher than the threshold, the instruction indicates that the subject has a high susceptibility to the allergic reaction. If the expression levels of NELL2 and AKAP12 and IL13RA1 and ITM2C of the tested subject are lower than the threshold, the instruction indicates that the subject has a low susceptibility to the allergic reaction.

In one example embodiment, the allergic reaction is Immunoglobulin E (IgE)-mediated allergic reaction. In one example embodiment, the mast cells are generated by an in-vitro method from the peripheral blood of the subject.

In one example embodiment, the tested subject does not need to be stimulated by an allergen when the kit is applied to determine the subject's susceptibility to the allergic reaction. This provides the subject with more convenience and flexibility to predict the likelihood of degree of severity of an allergic reaction.

In one example embodiment, the kit determines susceptibility to a severe allergic reaction. The severe allergic reaction includes a very strong reaction (with severe symptoms) to a certain allergen without significant health risk. The severe allergic reaction also includes a very strong reaction to a certain allergen with health risk or even risk of fatality, and such reaction is called anaphylaxis. The symptoms of the severe allergic reaction usually involve more than one organ system (one part of the body), such as the skin, mouth, eyes, lungs, heart, gut, and brain. The symptoms of severe allergic reaction include skin rashes, itching or hives; swelling of the lips, tongue or throat, shortness of breath, trouble breathing or wheezing (whistling sound during breathing); dizziness and/or fainting; stomach pain, vomiting or diarrhea; and feeling like something awful is about to happen, etc.

In one example embodiment, the kit determines susceptibility to severe IgE-mediated allergic reaction.

Example embodiments also provide a method of predicting susceptibility to an allergic reaction for a subject and treating or preventing the allergic reaction.

In one example embodiment, the method includes obtaining a biological sample from the subject. In one example embodiment, the biological sample is derived from blood of the subject. In one example embodiment, the biological sample is mast cells. In one example embodiment, the biological sample is blood-derived mast cells of the subject. In one example embodiment, the biological sample is mast cells generated by an in-vitro method from peripheral blood of the subject.

In one example embodiment, the method includes comparing an expression level of a target biomarker in the biological sample of the subject with a threshold. The method predicts the susceptibility of the subject to the allergic reaction by comparing the expression level of the target biomarker of the subject with a threshold. In some embodiments, one or more target biomarkers are used to predict the susceptibility of the subject to the allergic reaction. In some embodiments, one, two, three, or four target biomarkers are used. In one example embodiment, the target biomarkers are NELL2, AKAP12, IL13RA1, ITM2C or any combination thereof. In one example embodiment, the threshold is defined as the mean of expression levels of the target biomarker in the biological sample of subjects who have an average risk of allergic reaction.

In one example embodiment, the method includes identifying the subject as having a high susceptibility to the allergic reaction if the expression level of NELL2 and/or AKAP12 and/or IL13RA1 and/or ITM2C in the biological sample of the subject is higher than the threshold.

In one example embodiment, the threshold is defined as the mean of expression levels of the target biomarker in mast cells of subjects who have an average risk of the allergic reaction or whose mast cells exhibit normal average response to allergens. In one example embodiment, the threshold of NELL2 mRNA expression in mast cells generated from subjects whose blood-derived mast cells exhibit an average response of histamine release when activated via the high affinity IgE receptors is 1.88, which is the ratio of the copy numbers of NELL2 mRNA over copy numbers of GAPDH mRNA measured in a selected population of human mast cells using TaqMan qPCR probes and Quantitative PCR Human Reference Total RNA (Agilent cat. #750500) as the reference internal control.

In one example embodiment, the method includes identifying the subject as having a high susceptibility to the allergic reaction if the expression levels of NELL2 and AKAP12 and IL13RA1 and ITM2C in the mast cells of the subject are higher than the threshold.

In one example embodiment, the method includes identifying the subject as having a high susceptibility to the allergic reaction if the expression level of one of the target biomarkers or any combination of the target biomarkers in the biological sample of the subject is higher than the threshold. The method includes identifying the subject as having a low susceptibility to the allergic reaction if the expression level of one of the target biomarkers or any combination of the target biomarkers in the biological sample of the subject is lower than the threshold. The method includes identifying the subject as having an average susceptibility to the allergic reaction if the expression level of one of the target biomarkers of any combination of the target biomarkers in the biological sample of the subject is within the threshold.

In one example embodiment, the allergic reaction is severe allergic reaction. In one example embodiment, the allergic reaction is severe IgE-medicated allergic reaction.

In one example embodiment, the method further includes administering to the subject having the high susceptibility to the allergic reaction an anti-allergic drug for treatment or prevention of the allergic reaction. In one example embodiment, the method includes providing the subject having the high susceptibility a piece of information that the subject should avoid contacting an allergen in response to the allergic reaction for treatment or prevention of the allergic reaction. In one example embodiment, the method includes treating or preventing the subject having the high susceptibility by prescribing a diet or treatment regimen wherein the subject avoids contacting the allergen associated with the allergic reaction.

In one example embodiment, the method includes providing the subject with high susceptibility a piece of information that the subject needs to do more tests to find out the specific allergens the subject is allergic to. In one example embodiment, the method includes providing the subject with low susceptibility a piece of information that it may be not necessary for the subject to do more allergy diagnostics tests to find out the allergens which the subject may have been sensitized to.

Example embodiments also provide a method of predicting susceptibility to an allergic reaction for a subject and treating the allergic reaction.

In one example embodiment, the method includes the method of predicting susceptibility to an allergic reaction as discussed herein and a step of treating the subject with high susceptibility.

In one example embodiment, the method provides the subject with high susceptibility to the allergic reaction a treatment by administering to the subject an anti-allergic drug, or providing to the subject a piece of information that the subject should avoid contacting an allergen in response to the allergic reaction in the future, or provide to the subject a piece of suggestion that the subject should do more allergy diagnostics tests to find out the specific allergens which the subject may be sensitized and therefore react to.

Example embodiments also provide a kit for determining susceptibility to an allergic reaction for a subject. The kit includes a first probe for identifying AKAP12 from a biological sample of the subject, a second probe for identifying NELL2 from the biological sample of the subject, a third probe for identifying ITM2C from the biological sample of the subject, and a fourth probe for identifying IL13RA1 from the biological sample of the subject. The kit may include any one of the first, second, third and fourth probes, or any combination of these four probes.

The kit optionally includes an instruction that indicates the subject's susceptibility to the allergic reaction by comparing expression levels of NELL2, AKAP12, IL13RA1 and/or ITM2C in the biological sample of the subject with those who represents a threshold.

The threshold is defined as the mean of individual expression levels of NELL2, AKAP12, IL13RA1 and ITM2C in the biological sample of subjects who have an average risk of the allergic reaction.

In one example embodiment, the instruction indicates that the subject has a high susceptibility to the allergic reaction if the expression levels of NELL2 and AKAP12 and IL13RA1 and ITM2C are higher than the threshold; and the instruction indicates that the subject has a low susceptibility to the allergic reaction, if the expression levels of NELL2 and AKAP12 and IL13RA1 and ITM2C are lower than the threshold.

Example embodiments provide a method of identifying subjects having susceptibility to an allergic reaction. The method includes measuring the expression level of a target biomarker in a biological sample of the subjects; selecting out subjects whose expression levels of the target biomarker fit a certain profile. In some embodiments, the expression levels of one, two, three or four target biomarkers are measured and evaluated.

One example embodiment provides a method of identifying subjects having high susceptibility to an allergic reaction, wherein if the expression level of NELL2 and/or AKAP12 and/or IL13RA1 and/or ITM2C of the subject is higher than a threshold. One example embodiment provides a method of identifying subjects having low susceptibility to an allergic reaction, wherein if the expression level of NELL2 and/or AKAP12 and/or IL13RA1 and/or ITM2C of the subject is lower than the threshold.

The threshold is defined as the mean of individual expression levels of NELL2, AKAP12, IL13RA1, and/or ITM2C in the biological sample of subjects who have an average risk of allergic reaction.

The method also includes treating the subjects having high susceptibility to the allergic reaction by providing the subjects with an anti-allergic drug or prescribing a diet or treatment regimen wherein the subjects avoid exposing themselves to allergens that are associated with the allergic reaction.

Example embodiments also provide a method of diagnosing a patient with a potential allergy. The method includes comparing an expression level of NeLL2, AKAP12, ITM2C and/or IL13RA1 of the patient with a threshold, and diagnosing the patient as having the potential allergy if the expression level of NELL2 and/or AKAP12 and/or IL13RA1 and/or ITM2C is higher than the threshold. The threshold is defined as the mean of individual expression levels of the corresponding NELL2, AKAP12, ITM2C and/or IL13RA1 in subjects with an average risk of potential allergy.

In one example embodiment, a blood-based mast cell activation allergy test is provided for the identification/prediction of susceptibility of normal individuals and allergic patients to severe IgE-mediated allergic reactions in the context of Precision Medicine for personalized prevention and treatment. Using the identified functional genomics biomarkers that correlate with high level of mast cell activation in the effector phase of allergic reaction in-vitro, the test offers the direct prediction of the severity of potential allergic reaction in an individual in-vivo.

As used herein, the term “subject” is used herein in its broadest sense. Subjects may include isolated cells, either prokaryotic or eukaryotic, or tissues grown in culture. In certain embodiments, a subject is an animal, particularly an animal selected from a mammalian species including rat, rabbit, bovine, ovine, porcine, canine, feline, murine, equine, and primate, particularly human.

As used herein, the term “microarray” refers to any arrangement of nucleic acids, ammo acids, antibodies, etc., on a substrate. The substrate can be any suitable support, e.g., beads, glass, paper, nitrocellulose, nylon, or any appropriate membrane, etc. A substrate can be any rigid or semi-rigid support including, but not limited to, membranes, filters, wafers, chips, slides, fibers, beads, including magnetic or nonmagnetic beads, gels, tubing, plates, polymers, micro particles, capillaries, etc. The substrate can provide a surface for coating and/or can have a variety of surface forms, such as wells, trenches, channels, and pores, to which the nucleic acids, ammo acids, etc., may be bound.

As used herein, the term “biological sample” refers to a sample derived from any source where mast cells (e.g. connective tissue-type mast cells) exist, for example, from bodily fluids, secretions, tissues, cells, or cells in culture including, but not limited to, saliva, blood, urine, serum, plasma, vitreous, synovial fluid, cerebral spinal fluid, amniotic fluid, and organ tissue (such as biopsied tissue); from chromosomes, organelles, or other membranes isolated from a cell; from genomic DNA, cDNA, RNA, mRNA, etc.; and from cleared cells or tissues, or blots or imprints from such cells or tissues.

As used herein, the term “preventing” or “prevention” refers to a delay of onset, and/or reduction in frequency and/or severity of one or more symptoms of a particular disease, disorder and/or condition, such as allergy or allergic reaction. In some embodiments, prevention is assessed on a population basis such that a method or a kit is considered to “prevent” a particular disease, disorder and/or condition if a statistically significant decrease in the development, frequency, and/or intensity of one or more symptoms of the disease, disorder, and/or condition (e.g. allergy or allergic reaction) is observed in a population susceptible to the disease, disorder and/or condition. Prevention may be considered complete when onset of a disease, disorder and/or condition has been delayed for a predefined period of time.

As used herein, the term “treating” or “treatment” refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition (e.g. allergy or allergic reaction). In some embodiments, such treatment may be of a subject known to have one or more susceptibility factors that are statistically correlated with increased risk of development of the relevant disease, disorder, and/or condition.

As used herein, “anti-allergic drug” may be used synonymously with the terms “allergy drug”, “allergy medicine”, “allergy medication”, “anti-allergy medicine”, and refers to any sort of therapy that treats allergy or an allergic reaction, and include, but are not limited to, antihistamines, corticosteroids, mast cell stabilizers, leukotriene inhibitors, nasal anticholinergics, decongestants, immuno-modulators (immuno-suppressants), autoinjectable epinephrine, epinephrine, anti-IgE antibodies, anti-cytokine antibodies and anti-cytokine receptor antibodies.

### EXAMPLES

### Example 1 Generation of Primary Human Cultured Mast Cells In-Vitro

A protocol that can generate large numbers of mature human mast cells from individual buffy coat from human donors is employed using an in-vitro culturing system as described in U.S. Pat. No. 9,394,519, US publication 2013/0052676 or Issan Tam, et al., 2017 (A novel six-week protocol for generating functional human connective tissue-type (MC_{TC}) mast cells from buffy coats) published in Inflamm. Res. 2017. 66:25-37. U.S. Pat. No. 9,394,519, US publication 2013/0052676 and Issan Tam, et al., 2017 are hereby incorporated by reference in its entirety. These cultured human mast cells exhibit phenotypic and characteristic functional properties that are identical to those of human connective tissue-type mast cells located in the skin and other sites of allergic and inflammatory reactions.

### Example 2 Variations of Human Cultured Mast Cells in Levels of IgE-Dependent Functional Activation Among Different Individual Normal Donors

After assessing the functional activation of cultured mast cells derived from >50 human normal individual donors in response to different mast cell stimuli, significant differences in the levels and patterns of activation among these mast cell cultures in response to IgE/anti-IgE stimulation have been found. Specifically, primary human mast cell cultures derived from some individual donors responded very strongly to the stimulation, with their % histamine release equaled to twice of those of the average responders (˜25% histamine release). On the other hand, some individual donor cultures responded very poorly to the stimulation, with their % histamine release equaled to half of those of the average responders. The levels of functional activation in response to IgE-dependent stimulation vary significantly among individual human mast cell cultures that are derived from different normal individual human donors.

### Example 3 Human Cultured Mast Cell System as an Approach for Identification of Biomarkers for Assessing Susceptibility/Severity to Allergic Reactions in Individual Human Donors

Since mast cells are the major effector cells for allergic reactions, the individual variation in functional activation of mast cells to IgE-dependent stimulation in-vitro can represent a useful approach to identify biomarkers that can predict the susceptibility/severity of single individuals to allergic reactions in-vivo. Therefore, the functional genomics approach is used to identity genomics biomarkers that are correlated with the individual patterns of mast cell activation among different mast cell cultures. A signature biomarker or a signature set of genomics biomarkers are validated among a general human normal population as a diagnostic tool for predicting the susceptibility/severity of single human individuals to allergic reactions to different allergens such as proteins, foods, drugs and chemicals. These biomarkers are used as informative diagnostic tools in the practice of Precision Medicine to provide a personal genomics guide in predicting allergic reactions in (a) disease prevention for healthy individuals (b) patient stratification in clinical trials (c) personalized treatments for single individual patient.

### Example 4 Functional Genomics Profiling of Primary Human Mast Cell Cultures

Single normal individual donor primary human mast cell cultures are divided into the following 3 groups based on their responsiveness to anti-IgE stimulation mediated via the activation of the high-affinity IgE receptors in human mast cells: (a) High responsiveness (b) Low responsiveness (c) Average responsiveness. These cultures were processed for preparation of RNA samples, which were further processed for mRNA expression analyses using microarray and TaqMan qPCR.

### Example 5 Methods

1. Generation and characterization of single normal donor primary human mast cell cultures

A. Sample acquisition workflow:

a) Single human normal donor buffy coats are obtained from Hong Kong Red Cross.

b) CD34⁺ progenitors isolation is performed on Day 1.

c) Human cultured mast cells with connective tissue-type phenotypes (with expression of both chymase and tryptase) are generated in-vitro using the primary human mast cell culturing protocol as described U.S. Pat. No. 9,394,519, US publication 2013/0052676 and Issan Tam et al., 2017.

d) Chymase and tryptase staining are performed on week 9.

e) Histamine release (%) as an index of functional activation of human cultured mast cells is performed on week 9. The method for measuring histamine release is described in detail in Issan Tam et al., 2017.

f) Extraction of RNA using RNAiso Plus (Takara) is performed on week 9.

B. Function activation profiling:

Profiles for chymase/tryptase expression and for % histamine release induced by 2 different stimuli (anti-IgE, A23187) for each donor are generated.

C. Functional genomics approaches:

RNA samples: the functional activation induced by the stimulation of individual mast cell cultures by anti-IgE (i.e. mast cell activation mediated by the activation of the high affinity IgE receptors) is assessed. By calculating the mean+/−one standard deviation, the responding mast cell cultures are categorized into 3 different populations according to the levels of % histamine release induced by anti-IgE treatment: High responders, Low responders, Average responders. RNA is subsequently extracted from these mast cell cultures.

2. Functional genomics profiling:

RNA samples extracted from 50 human mast cell cultures described above (i.e. representing 19 High responders, 14 Low responders, and 17 Average responders) are subjected to microarray analysis as the primary screening.

Microarray analysis is performed using Affymetrix GeneChip Human Gene 2.0 ST Array and data are analyzed using the Partek Genomics Suite software.

Four genes are identified by microarray analysis as the potential signature genes whose expressions show direct correlations with the functional activation profiles of those mast cell cultures from which they are derived. Expression levels of these four genes are further validated using TaqMan real-time qPCR on some of the 50 original human mast cell cultures and on some other mast cell cultures that are derived from different normal individual donors. The pattern or signature of the validated gene expression that are correlated with different levels of mast cell activation are designated as the biomarkers for predicting the susceptibility of single normal individuals to severe allergic reactions.

### Example 6 Results

1. Establishment of single normal donor human mast cell cultures

146 single donor primary human mast cell cultures were generated using the culturing protocol as described in U.S. Pat. No. 9,394,519 and in Issan Tam et al., 2017.

2. Collection of data for the functional categorization of individual human mast cell cultures by assaying their responses to anti-IgE and other mast cell activators

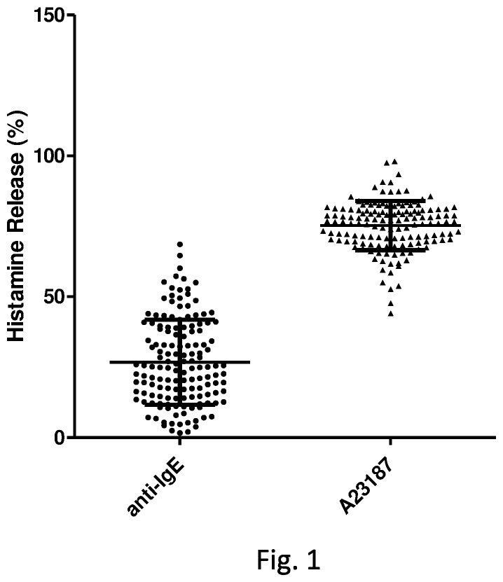

Functional data were collected from 146 single donor primary human mast cell cultures. Details of the individual cultures are shown in FIG. 1. FIG. 1 shows that histamine release (%) of 146 mast cell cultures stimulated by anti-IgE and A23187, respectively.

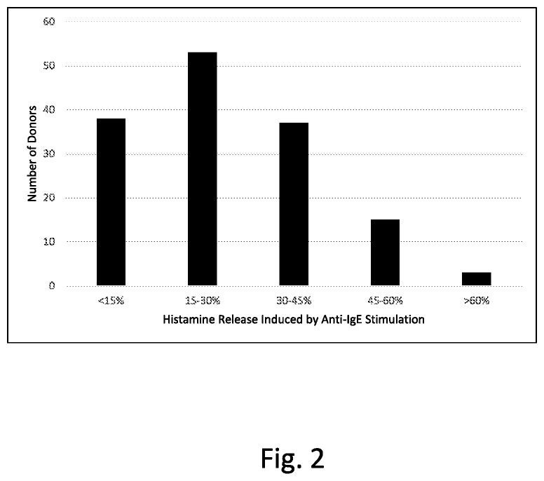

Distribution of numbers of donor cultures with different respective levels of degranulation response to anti-IgE stimulation (graphed in 15% intervals) showed a peak at 15%-30% (N=53) (FIG. 2). In the 146-donor cohort, mean % histamine release was 26.8% with a standard deviation of ±15.1%. Therefore, the range for “normal average response” among the selected donor population was assigned as 11.7%-41.9% (Average responders, N=95). For donors with responses <11.7% were designated as Low responders (N=24), whereas those with responses >41.9% were designated as High responders (N=27).

Furthermore, by screening 60 of the 146 selected donors, it was found that more than 96% of the cells in each donor sample were stained positively for expression of specific mast cell marker tryptase, thus suggesting that these cultures were indeed mature and homogenous human mast cells.

Selection of donor samples for microarray analysis as a primary screening for identification of functional genomics markers

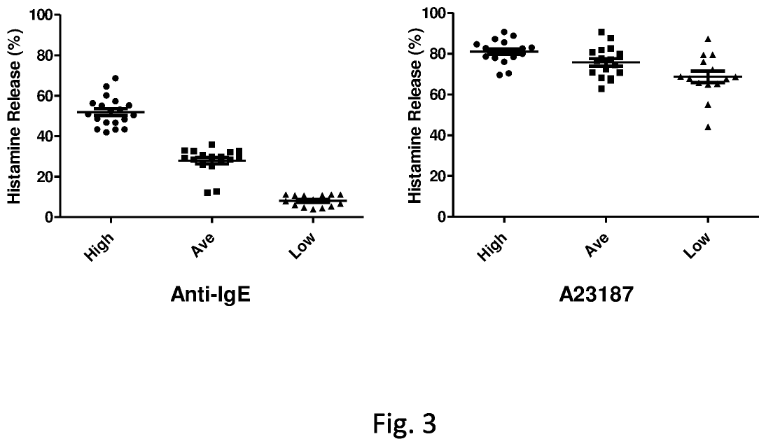

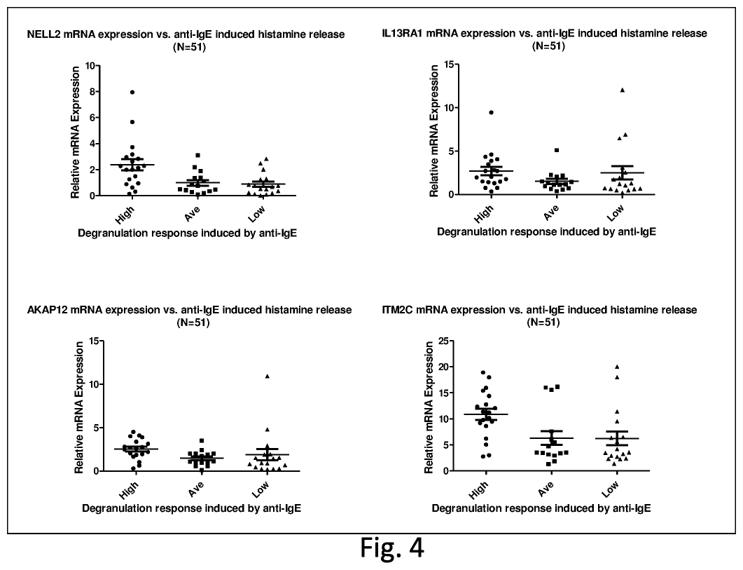

Unstimulated samples (total N=50) derived from each of the High/Average/Low responder groups were selected for microarray analysis according to the corresponding histamine release data generated from the 146-donor cohort shown above. Donor samples were picked from High (% release>41.9%, N=19), Low (% release<11.7%, N=14), and Average (% release between 11.7% and 41.9%, N=17) responder groups. FIG. 3 shows the level of % histamine release in response to either IgE or A23137 stimulation for each sample of the three respective responder groups that was comprised in the 50 donor samples selected.

In the primary screening using microarray analysis, NELL2 and AKAP12 and IL13RA1 and ITM2C were identified as genes whose expression levels were up-regulated in High responders compared to other responder groups.

4. TaqMan qPCR assay

A signature gene or a signature multi-gene set whose expression pattern can predict the susceptibility of an individual to severe allergic reaction is developed in the assay.

TaqMan qPCR assay was performed for four genes, i.e. AKAP12 (A-kinase anchor protein 12) and NELL2 (neural EGFL like 2 protein) and ITM2C (integral membrane protein 2C) and interleukin 13 receptor subunit alpha 1 (IL13RA1) using cDNAs that were reverse transcribed from total RNAs derived from selected donor samples. Data analysis of TaqMan qPCR studies (calculated as 2\hat{ }-ddCt) showed a significant correlation of levels of % histamine release with gene expression levels of these 4 genes, suggesting that the expressions of NELL2, AKAP12, IL13RA1, and ITM2C genes in basal unstimulated states can differentiate the High responders from the Average responders in the human cultured mast cell cultures. FIG. 4 shows significant difference in the expression levels of NELL, AKAP12, IL13RA1, and ITM2C between High responder group vs Average responder group. Therefore, NELL2, AKAP12, ITM2C and IL13RA1 can be employed as the representative genes in the multi-gene set whose signature expression in primary human cultured mast cells in-vitro can predict high level of mast cell activation and subsequently high allergic reactions to allergen sensitization in a single individual in-vivo.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

What is claimed is:

1. A kit for determining susceptibility to an allergic reaction for a subject, comprising:
(a) a probe for identifying a target biomarker from a biological sample of the subject; and
(b) an instruction that indicates the subject's susceptibility to the allergic reaction by comparing an expression level of the target biomarker in the biological sample of the subject with a threshold,
wherein the target biomarker is selected from a group consisting of neural EGFL like 2 protein (NELL2), A-kinase anchor protein 12 (AKAP12), integral membrane protein 2C (ITM2C), interleukin 13 receptor subunit alpha 1 (IL13RA1) and any combination thereof,
wherein the threshold is defined as the mean of expression levels of the target biomarker in the biological samples of subjects who have an average risk of undergoing an allergic reaction.

2. The kit of claim 1, wherein the instruction indicates that the subject has a high susceptibility to the allergic reaction if the expression level of the target biomarker of the subject is higher than the threshold; and the subject has a low susceptibility to the allergic reaction if the expression level of the target biomarker of the subject is lower than the threshold.

3. The kit of claim 2, wherein the instruction indicates that the subject has an average susceptibility to the allergic reaction if the expression level of the target biomarker of the subject is within the threshold.

4. The kit of claim 1, wherein the allergic reaction is a severe Immunoglobulin E (IgE)-mediated allergic reaction.

5. The kit of claim 1, wherein the biological sample is derived from blood of the subject.

6. The kit of claim 1, wherein the biological sample is mast cells generated by an in-vitro method from peripheral blood of the subject.

7. The kit of claim 1, wherein the subject does not need to be stimulated by an allergen when the kit is applied to determine the subject's susceptibility to the allergic reaction.

8. The kit of claim 1, wherein the threshold is defined by the following steps:
determining levels of histamine release in response to an allergen in a group of sample subjects;
calculating a mean value of the levels of the histamine release among the group of sample subjects and optionally a standard deviation value;
calculating a range for an average histamine release, wherein the range is defined as [mean value−standard deviation value] (lower limit) to [mean value+standard deviation value] (upper limit)
defining the mean of expression levels of the target biomarker in the biological sample from subjects with the average histamine release as the threshold.

9. A method of treating or preventing an allergic reaction in a subject, comprising:
obtaining a biological sample from the subject;
comparing an expression level of a target biomarker in the biological sample of the subject with a threshold,
wherein the target biomarker is selected from a group consisting of neural EGFL like 2 protein (NELL2), A-kinase anchor protein 12 (AKAP12), integral membrane protein 2C (ITM2C), interleukin 13 receptor subunit alpha 1 (IL13RA1) and any combination thereof; and
wherein the threshold is defined as the expression level of the target biomarker in the biological sample of a subject who has an average risk of the allergic reaction,
identifying the subject as having a high susceptibility to the allergic reaction if the expression level of the target biomarker in the biological sample of the subject is higher than the threshold; and
administering to the subject having the high susceptibility an anti-allergic drug for treatment or prevention of the allergic reaction, or
treating or preventing the subject having the high susceptibility by prescribing a diet or treatment regimen wherein the subject avoids contacting an allergen associated with the allergic reaction.

10. The method of claim 9, wherein the allergic reaction is severe allergic reaction.

11. The method of claim 9, wherein the allergic reaction is severe Immunoglobulin E (IgE)-mediated allergic reaction.

12. The method of claim 9, wherein the allergic reaction is IgE-mediated allergic reaction.

13. The method of claim 9, wherein the biological sample is derived from blood of the subject.

14. The method of claim 9, wherein the biological sample is mast cells generated by an in-vitro method from peripheral blood of the subject.

15. The method of claim 9, wherein the threshold is obtained by calculating the mean of expression levels of the target biomarker in the biological sample of subjects who have an average risk of allergic reaction.

16. The method of claim 9, wherein the threshold for the average risk of the allergic reaction is defined according to the following steps:
determining levels of histamine release in response to an allergen in a group of sample subjects;
calculating a mean value of the levels of the histamine release among the group of sample subjects and optionally a standard deviation value;
calculating a range for an average histamine release, wherein the range is defined as [mean value−standard deviation value] (lower limit) to [mean value+standard deviation value] (upper limit);
defining the mean of the expression levels of the target biomarker in biological sample of subjects with the average histamine release as the threshold.

17. The method of claim 9, wherein if the expression level of the target biomarker of the subject is within the threshold, the subject has an average likelihood of the allergic reaction; and if the expression level of the target biomarker of the subject is lower than the threshold, the subject has a low likelihood of the allergic reaction.

18. A kit for determining susceptibility to an allergic reaction for a subject, comprising:
a first probe for identifying A-kinase anchor protein 12 (AKAP12) from a biological sample of the subject;
a second probe for identifying neural EGFL like 2 protein (NELL2) from the biological sample of the subject;
a third probe for identifying integral membrane protein 2C (ITM2C) from the biological sample of the subject; and
a fourth probe for identifying interleukin 13 receptor subunit alpha 1 (IL13RA1) from the biological sample of the subject.

19. The kit of claim 18 further comprising:
an instruction that indicates the subject's susceptibility to the allergic reaction by comparing expression levels of NELL2, AKAP12, IL13RA1 and ITM2C in the biological sample of the subject with a threshold,
wherein the threshold is defined as the mean of the individual expression levels of NELL2, AKAP12, IL13RA1 and ITM2C in the biological sample of subjects who have an average risk of allergic reaction.

20. The kit of claim 19, wherein the instruction indicates that the subject has a high susceptibility to the allergic reaction if the expression levels of NELL2, AKAP12 and IL13RA1 and ITM2C are higher than the threshold; and the instruction indicates that the subject has a low susceptibility to the allergic reaction if the expression levels of NELL2 and AKAP12 and IL13RA1 and ITM2C are lower than the threshold.
